(19) United States
(12) Patent Application Publication
(10) Pub. No.: US 2011/0280806 A1
(43) Pub. Date: Nov. 17, 2011

(54) DYE CONJUGATE IMAGING AGENTS

(76) Inventors: Robert James Domett Nairne, Amersham (GB); Alexander Jackson, Amersham (GB)

(21) Appl. No.: 13/130,075

(22) PCT Filed: Nov. 19, 2009
(86) PCT No.: PCT/EP09/65461

§ 371 Date: May 19, 2011

(30) Foreign Application Priority Data
Nov. 20, 2008 (EP) 08169526.4

(51) Int. Cl. A61K 49/00; C07H 21/02; C07H 21/04; C07K 4/00; C07D 209/86; C07K 14/00

(52) U.S. Cl. 424/9.1; 548/449; 530/300; 536/23.1

(57) ABSTRACT

The present invention relates to imaging agents suitable for in vivo optical imaging, which comprise conjugates of dihydrocarbazolium dyes with biological targeting moieties, such as peptides. Also disclosed are pharmaceutical compositions and kits, as well as in vivo imaging methods. The dihydrocarbazolium dyes are functionalised with water solubilising groups and have functional groups which facilitate conjugation to biological targeting moieties.

### FIELD OF THE INVENTION

The present invention relates to imaging agents suitable for in vivo optical imaging, which comprise conjugates of dihydrocarbazolium dyes with biological targeting moieties, such as peptides. Also disclosed are pharmaceutical compositions and kits, as well as in vivo imaging methods.

### BACKGROUND TO THE INVENTION

Many optical imaging agents use near-infra red (NIR) absorbing cyanine dyes as their fluorescent optical reporter. To maximise sensitivity of the optical signal, the optimum photophysical properties of the dye should allow excitation and emission in the part of the electromagnetic spectrum at which tissue is at its most transparent—between 700 and 850 nm. Cyanine dyes that meet this requirement are heptamethine cyanine dyes (Formula A):

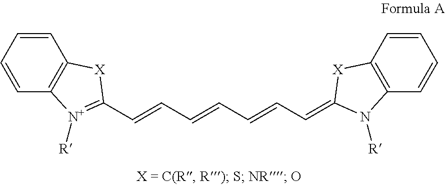

Due to the length of the polymethine chain, heptamethine cyanine dyes exhibit reduced chemical and photostability over the shorter wavelength-absorbing penta- and trimethine cyanines. For in vivo applications, there have been attempts to increase the stability of heptamethine cyanine dyes by forming rings in the centre of the methine chain.

U.S. Pat. No. 6,083,485 and counterparts discloses in vivo near-infrared (NIR) optical imaging methods using cyanine dyes having an octanol-water partition coefficient of 2.0 or less. Also disclosed are conjugates of said dyes with “biological detecting units” of molecular weight up to 30 kDa which bind to specific cell populations, or bind selectively to receptors, or accumulate in tissues or tumours. The dyes of U.S. Pat. No. 6,083,485 may also be conjugated to macromolecules, such as polylysine, dextran or polyethylene glycol. No specific dye-conjugates are disclosed.

U.S. Pat. No. 5,892,056 discloses dyes of Formula B:

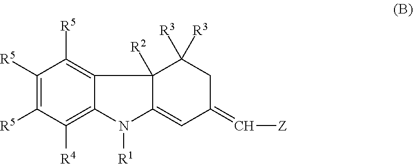

wherein:
R¹ is C₁₋₁₈ alkyl, aryl, sulfoalkyl, carboxyalkyl, sulfatoalkyl, acyloxyalkyl, dialkylaminoalkylene, cycloaminoalkylene, acyl or alkenyl;
R² is C₁₋₁₈ alkyl;
R³ and R⁴ are H or C₁₋₁₈ alkyl;
R⁵ is H, NO₂, carboxyl, sulfo, OH, Hal, phospho; or C₁₋₁₈ alkoxy, thioalkoxy, oxyalkyl, acyl, alkyl, aryl or amino group,
   wherein any two R⁵ groups, or R⁴ and R⁵, or R¹ and R⁴ may together form a substituted or unsubstituted aryl, heteroaryl, aliphatic or heterocyclic ring; and
Z represents the atoms necessary to complete a dye selected from the group consisting of: carbocyanine, azacarbocyanine, hemicyanine, styryl, diazacarbocyanine, triazacarbocyanine, diazahemicyanine, polymethinecyanine, azapolymethinecyanine, holopolar, indocyanine, merocyanine, squarilium and diazahemicyanine dyes.

U.S. Pat. No. 5,892,056 does not disclose conjugates of the dyes with biological targeting moieties or functionalised versions of the dyes suitable for preparing such conjugates. Nor does U.S. Pat. No. 5,892,056 disclose in vivo optical imaging applications.

JP 2005-220045 A(Konica Minolta MG KK) discloses dyes encapsulated within microcarriers, particularly liposomes, for in vivo optical imaging—especially of cancer. The dyes described are cyanine dyes, including indocyanine green (ICG), and dyes disclosed in U.S. Pat. No. 5,892,056, including such dyes having at least four sulfonate group substituents. JP 2005-220045 does not disclose conjugates of any of the dyes therein with biological targeting molecules.

US 2005/0136007 A1 discloses a near infra-red fluorescent contrast medium which comprises a cyaninc compound of Formula C:

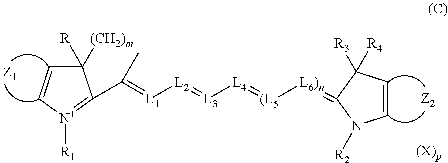

wherein:
R is H, a lower alkyl group or an aromatic group;
R₁ and R₂ are each an aliphatic group containing a water-solubilising group;
R₃ and R₄ are each a lower alkyl group or an aromatic group, provided that R₃ and R₄ may combine with each other to form a carbocyclic ring; L₁ to L₆ are each a methine group, provided that when n is 1 or 2, L₆ may combine with R₃ or R₄ to form a carbocyclic ring and when n is 0, L₄ may combine with R₃ or R₄ to form a carbocyclic ring;
Z₁ and Z₂ are each a non-metallic atom group necessary to form a 5-or 6-membered ring;
X is a counter ion necessary to neutralize a charge of the molecule;
p is the number of X necessary to neutralize a charge of the molecule;
m is an integer of 2 to 4; and
n is an integer of 0 to 2.

US 2005/0136007 A1 does not disclose conjugates of the dyes with biological targeting moieties or functionalised versions of the dyes suitable for preparing such conjugates.

### The Present Invention.

The present invention provides dihydrocarbazolium dyes, which have photophysical properties suitable for optical imaging in vivo. The dyes of the invention have been found to be fluorescent, and to have properties comparable to the cyanine dye Cy7 This fluorescent property was not reported for dihydrocarbazolium dyes in the prior art.

The dihydrocarbazolium dyes of the present invention have 2 carbon atoms of the methine chain linking the heterocyclic rings forming part of the 6-membered ring. The dyes of US 2005/0136007 A1 have only one carbon atom of the methine chain forming part of a fused ring. The dyes of the present invention are functionalised with water solubilising groups and groups which facilitate conjugation to biological targeting molecules. That renders the dyes of the invention useful for optical imaging in vivo as conjugates with a variety of biological targeting molecules. The dyes of the present invention also have a higher quantum yield than the corresponding heptamethine cyanine dyes.

### DETAILED DESCRIPTION OF THE INVENTION

In a first aspect, the present invention provides an imaging agent suitable for in vivo optical imaging of the mammalian body which comprises a conjugate of Formula I:

[BTM]-(L)ₙ-Cz^{D} (I)

where:
BTM is a biological targeting moiety;
CZ^{D} is a dihydrocarbazolium dye of Formula II:

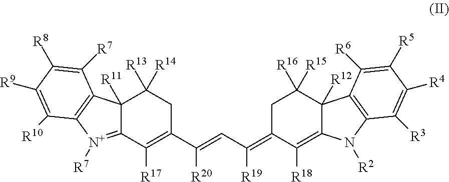

where:
R¹, R², and R¹¹ to R¹⁶ are each independently R^{a} groups,
R³ to R¹⁰ are each independently H, —SO₃M¹,
   —CO₂M¹, C₂₋₇ carboxyalkyl, C₁₋₄ hydroxyalkyl, or C₂₋₇ carboxamidoalkyl optionally substituted with 1 to 3 hydroxy groups, where M¹ is independently H or B^{c}, and B^{c} is a biocompatible cation;
R¹⁷ to R²⁰ are each independently H or an R^{a} group;
where R^{a} is C₁₋₄ alkyl, C₁₋₄ sulfoalkyl, C₂₋₇ carboxyalkyl or C₁₋₄ hydroxyalkyl;
L is a synthetic linker group of formula -(A)ₘ- wherein each A is independently —CR₂—, —CR═CR—, —C═C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
n is an integer of value 0 or 1;
   with the proviso that the dihydrocarbazolium dye comprises at least 2 sulfonic acid substituents.

By the term “imaging agent” is meant a compound suitable for optical imaging of a region of interest of the whole (ie. intact) mammalian body in viva Preferably, the mammal is a human subject. The imaging may be invasive (eg. intra-operative or endoscopic) or non-invasive. The imaging may optionally be used to facilitate biopsy (eg. via a biopsy channel in an endoscope instrument), or tumour resection (eg. during intra-operative procedures via tumour margin identification).

Whilst the conjugate of Formula I is suitable for in vivo imaging, it may also have in vitro applications (eg. assays quantifying the BTM in biological samples or visualisation of BTM in tissue samples). Preferably, the imaging agent is used for in vivo imaging.

By the term “biological targeting moiety” (BTM) is meant a compound which, after administration, is taken up selectively or localises at a particular site of the mammalian body. Such sites may for example be implicated in a particular disease state be indicative of how an organ or metabolic process is functioning. The biological targeting moiety preferably comprises: a 3-100 mer peptide, peptide analogue, peptoid or peptide mimetic which may be a linear or cyclic peptide or combination thereof; a single amino acid, an enzyme substrate, enzyme antagonist enzyme agonist (including partial agonist) or enzyme inhibitor; receptor-binding compound (including a receptor substrate, antagonist, agonist or substrate); oligonucleotides, or oligo-DNA or oligo-RNA fragments.

By the term “peptide” is meant a compound comprising two or more amino acids, as defined below, linked by a peptide bond (ie. an amide bond linking the amine of one amino acid to the carboxyl of another). The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. The term “peptide analogue” refers to peptides comprising one or more amino acid analogues, as described below. See also “Synthesis of Peptides and Peptidomimetics”, M. Goodman et al, Houben-Weyl E22c, Thieme.

By the term “amino acid” is meant an L- or D-amino acid, amino acid analogue (eg. naphthylalanine) or amino acid mimetic which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure. By the term “amino acid mimetic” is meant synthetic analogues of naturally occurring amino acids which are isosteres, i.e. have been designed to mimic the steric and electronic structure of the natural compound. Such isosteres are well known to those skilled in the art and include but are not limited to depsipeptides, retro-inverso peptides, thioamides, cycloalkanes or 1,5-disubstituted tetrazoles [see M. Goodman, Biopolymers, 24, 137, (1985)].

When the BTM is an enzyme substrate, enzyme antagonist, enzyme agonist, enzyme inhibitor or receptor-binding compound it is preferably a non-peptide, and more preferably is synthetic. By the term “non-peptide” is meant a compound which does not comprise any peptide bonds, ie. an amide bond between two amino acid residues. Suitable enzyme substrates, antagonists, agonists or inhibitors include glucose and glucose analogues such as fluorodeoxyglucose; fatty acids, or elastase, Angiotensin II or metalloproteinase inhibitors. A preferred non-peptide Angiotensin H antagonist is Losartan. Suitable synthetic receptor-binding compounds include estradiol, estrogen, progestin, progesterone and other steroid hormones; ligands for the dopamine D-1 or D-2 receptor, or dopamine transporter such as tropanes; and ligands for the serotonin receptor.

By the term “sulfonic acid substituent” is meant a substituent of formula —SO₃M¹, where M¹ is H or B^{c}, and B^{c} is a biocompatible cation. The —SO₃M¹, substituent is covalently bonded to a carbon atom, and the carbon atom may be aryl (such as the R³ to R¹⁰ groups), or alkyl (ie. a sulfoalkyl group). By the term “biocompatible cation” (B^{c}) is meant a positively charged counterion which forms a salt with an ionised, negatively charged group (in this case a sulfonate group), where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

The dihydrocarbazolium dye (Cz^{D}) of Formula II is a fluorescent dye or chromophore which is capable of detection either directly or indirectly in an optical imaging procedure using light of green to near-infrared wavelength (500-1200 nm, preferably 600-1000 nm). Preferably, the Cz^{D} has fluorescent properties.

It is envisaged that one of the roles of the linker group -(A)ₘ- of Formula I is to distance the Cz^{D} from the active site of the BTM. This is particularly important because the Cz^{D} is relatively bulky, so adverse steric interactions are possible. This can be achieved by a combination of flexibility (eg. simple alkyl chains), so that the Cz^{D} has the freedom to position itself away from the active site and/or rigidity such as a cycloalkyl or aryl spacer which orientate the Cz^{D} away from the active site. The nature of the linker group can also be used to modify the biodistribution of the imaging agent. Thus, eg. the introduction of ether groups in the linker will help to minimise plasma protein binding. When -(A)ₘ- comprises a polyethyleneglycol (PEG) building block or a peptide chain of 1 to 10 amino acid residues, the linker group may function to modify the pharmacokinetics and blood clearance rates of the imaging agent in vivo. Such “biomodifier” linker groups may accelerate the clearance of the imaging agent from background tissue, such as muscle or liver, and/or from the blood, thus giving a better diagnostic image due to less background interference. A biomodifier linker group may also be used to favour a particular route of excretion, eg. advantage that their manufacture and impurity profile can be fully controlled. Monoclonal antibodies and fragments thereof of natural origin are therefore outside the scope of the term ‘synthetic’ as used herein. The BTM is preferably chosen from: a 3-100 mer peptide, enzyme substrate, enzyme antagonist or enzyme inhibitor. BTM is most preferably a 3-100 mer peptide or peptide analogue. When the BTM is a peptide, it is preferably a 4-30 mer peptide, and most preferably a 5 to 28-mer peptide.

In Formula II, preferably at least one of R¹⁹ and R²⁰ is H, most preferably both are H.

The carboxamidoalkyl substituent of R³ to R¹⁰ is preferably of formula

—(CHR′)ₓCONR⁴⁰₂
where x is an integer of value 1 to 6, and
each R′ is independently H, C₁₋₃ alkyl or C₁₋₃ hydroxyalkyl.

A preferred such substituent is —(CH₂)ₓCONR′₂, where x and R′ are as defined above.

The dihydrocarbazolium dye (Cz^{D}) preferably has a total of 3 or 4 sulfonic acid substituents chosen from the —SO₃M¹ groups (of R³ to R¹⁰) and the sulfoalkyl groups (when R^{a} is chosen to be C₁₋₄ sulfoalkyl). Preferably, Cz^{D} comprises 1 to 3 sulfoalkyl substituents, most preferably at least 2 of the sulfonic acid substituents of Cz^{D} are chosen to be sulfoalkyl groups. The sulfoalkyl groups are preferably located at positions R¹, R², R¹⁵ or R¹⁸. In Formula II, the sulfoalkyl groups are preferably of formula —(CH₂)ₖSO₃M¹, where M¹ is H or B^{c}, k is an integer of value 1 to 4, and B^{c} is a biocompatible cation (as defined above). k is preferably 3 or 4.

R¹¹ and R¹² in Formula II are preferably chosen such that one is an R^{b} group, and the other is CH₃, where R^{b} is C₁₋₄ sulfoalkyl or C₂₋₇ carboxyalkyl.

The [BTM]-(L)ₙ- moiety of Formula I is preferably attached at positions R¹, R², R¹¹, or R¹⁷ of the Cz^{D} of Formula II, more preferably at R¹, R¹¹ or R¹⁷, most preferably at R¹ or R¹¹.

Especially preferred Cz^{p} dyes are of Formula IIa:

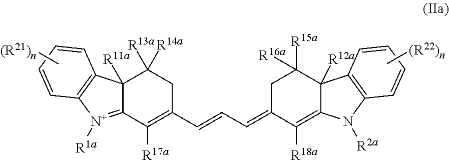

where:
R^{1a} and R^{2a} are each independently R^{b} groups;
R^{11a} to R^{12a} are each independently CH₃ or an R^{b} group;
R^{13a} to R^{15a} are each independently CH₃, CH₂OH or C₂₋₅ carboxyalkyl;
R^{17a} and R^{18a} are each independently H or an R^{b} group;
R²¹ and R²² are each independently —SO₃M¹ or —CO₂M¹;
where R^{b} and M¹ are as defined above;
each n is independently 0, 1 or 2.

In Formula IIa, preferably at least one of R^{17a} and R^{18a} is H. Most preferably, R^{17a}=R^{18a}=H. In Formula IIa, preferably at least one of R^{1a} and R^{2a} is C₁₋₄ sulfoalkyl, most preferably both are C₁₋₄ sulfoalkyl. In Formula IIa, preferably one of R^{11a} and R^{12a} is an R^{b} group, and the other is CH₃, where R^{b} is as defined above.

When the BTM is a peptide, preferred such peptides include:
somatostatin, octreotide and analogues,
peptides which bind to the ST receptor, where ST refers to the heat-stable toxin produced by E.coli and other micro-organisms;
laminin fragments eg. YIGSR, PDSGR, IKVAV, LRE and KCQAGTFALRGDPQG,
N-formyl peptides for targeting sites of leucocyte accumulation,
Platelet factor 4 (PF4) and fragments thereof,
RGD (Arg-Gly-Asp)-containing peptides, which may eg. target angiogenesis [R.Pasqualini et al., Nat Biotechnol. 1997 Jun; 15(6):542-6]; [E. Ruoslahti, Kidney Int. 1997 May; 51(5):1413-7].
peptide fragments of α₂-antiplasmin, fibronectin or beta-casein, fibrinogen or thrombospondin. The amino acid sequences of α₂-antiplasmin, fibronectin, beta-casein, fibrinogen and thrombospondin can be found in the following references: α₂-antiplasmin precursor [M.Tone et al., J.Biochem, 102, 1033, (1987)]; beta-casein [L.Hansson et al, Gene, 139, 193, (1994)]; fibronectin [A.Gutman et al, FEBS Lett., 207, 145, (1996)]; thrombospondin-1 precursor [V.Dixit et al, Proc. Natl. Acad. Sci., USA, 83, 5449, (1986)]; R. F.Doolittle, Ann. Rev. Biochem., 53, 195, (1984);
peptides which are substrates or inhibitors of angiotensin, such as: angiotensin II Asp-Arg-Val-Tyr-Ile-His-Pro-Phe (E. C. Jorgensen et al, J. Med. Chem., 1979, Vol 22, 9, 1038-1044) [Sar, Ile] Angiotensin II: Sar-Arg-Val-Tyr-Ile-His-Pro-Ile (R. K. Turker et al., Science, 1972, 177, 1203).
Angiotensin I: Asp-Arg-Val-Tyr-Ile-His-Pro-Phe-His-Leu.

When the BTM is a peptide, one or both termini of the peptide, preferably both, have conjugated thereto a metabolism inhibiting group (M^{IG}). Having both peptide termini protected in this way is important for in vivo imaging applications, since otherwise rapid metabolism would be expected with consequent loss of selective binding affinity for the BTM peptide. By the term “metabolism inhibiting group” (M^{IG}) is meant a biocompatible group which inhibits or suppresses enzyme, especially peptidase such as carboxypeptidase, metabolism of the BTM peptide at either the amino terminus or carboxy terminus. Such groups are particularly important for in vivo applications, and are well known to those skilled in the art and are suitably chosen from, for the peptide amine terminus:

N-acylated groups —NH(C═O)R^{G} where the acyl group —(C50 O)R^{G} has R^{G} chosen from: C₁₋₆ alkyl, C₃₋₁₀ aryl groups or comprises a polyethyleneglycol (PEG) building block. Suitable PEG groups are described for the linker group (L), below. Preferred such PEG groups are the biomodifiers of Formulae Bio1 or Bio2 (below). Preferred such amino terminus M^{IG} groups are acetyl, benzyloxycarbonyl or trifluoroacetyl, most preferably acetyl.

Suitable metabolism inhibiting groups for the peptide carboxyl terminus include: carboxamide, tert-butyl ester, benzyl ester, cyclohexyl ester, amino alcohol or a polyethyleneglycol (PEG) building block. A suitable M^{IG} group for the carboxy terminal amino acid residue of the BTM peptide is where the terminal amine of the amino acid residue is N-alkylated with a C₁₋₄ alkyl group, preferably a methyl group. Preferred such M^{IG} groups are carboxamide or PEG, most preferred such groups are carboxamide.

When either or both peptide termini are protected with an M^{1G} group, the -(L)ₙ[Cz^{D}] moiety may optionally be attached to the M^{IG} group. Preferably, at least one peptide terminus has no M^{IG} group, so that attachment of the -(L)ₙ[Cz^{D}] moiety at that position gives compounds of Formulae IVa or IVb respectively:

[Cz^{D}]-(L)ₙ-[BTM]-Z² (IVa);

Z¹-[BTM]-(L)ₙ-[Cz^{D}] (IVb);

where:n
Z¹ is attached to the N-terminus of the BTM peptide, and is H or M^{IG};
Z² is attached to the C-terminus of the BTM peptide and is OH, OB^{c}, or M^{IG}, where B^{c} is a biocompatible cation (as defined above).

In Formula IVa and IVb, Z¹ and Z² are preferably both independently M^{IG}. Preferred such M^{IG} groups for Z¹ and Z² are as described above for the peptide termini. Whilst inhibition of metabolism of the BTM peptide at either peptide terminus may also be achieved by attachment of the -(L)ₙ[Cz^{D}] moiety in this way, -(L)ₙ[Cz^{D}] itself is outside the definition of M^{IG} of the present invention.

The BTM peptide may optionally comprise at least one additional amino acid residue which possesses a side chain suitable for facile conjugation of the Cz^{D}, and forms part of the A residues of the linker group (L). Suitable such amino acid residues include Asp or Glu residues for conjugation with amine-functionalised Cz^{D} dyes, or a Lys residue for conjugation with a carboxy- or active ester- functionalised Cz^{D} dye. The additional amino acid residue(s) for conjugation of Cz^{D} are suitably located away from the binding region of the BTM peptide, and are preferably located at either the C- or N-terminus. Preferably, the amino acid residue for conjugation is a Lys residue.

When a synthetic linker group (L) is present, it preferably comprises terminal functional groups which facilitate conjugation to [BTM] and Cz^{D}. Suitable such groups (Q^{a}) are described in the fifth aspect (below). When L comprises a peptide chain of 1 to 10 amino acid residues, the amino acid residues are preferably chosen from glycine, lysine, arginine, aspartic acid, glutamic acid or serine. When L comprises a PEG moiety, it preferably comprises units derived from oligomerisation of the monodisperse PEG-like structures of Formulae Bio1 or Bio2:

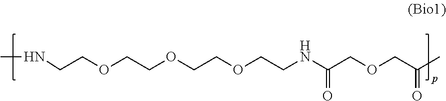

17-amino-5-oxo-6-aza-3, 9, 12, 15-tetraoxaheptadecanoic acid of Formula Bio1
wherein p is an integer from 1 to 10. Alternatively, a PEG-like structure based on a propionic acid derivative of Formula Bio2 can be used:

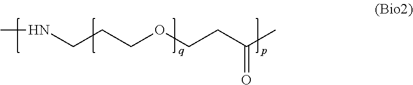

where p is as defined for Formula Bio1
and q is an integer from 3 to 15.

In Formula Bio2, p is preferably 1 or 2, and q is preferably 5 to 12.

When the linker group does not comprise PEG or a peptide chain, preferred L groups have a backbone chain of linked atoms which make up the -(A)ₘ- moiety of 2 to 10 atoms, most preferably 2 to 5 atoms, with 2 or 3 atoms being especially preferred. A minimum linker group backbone chain of 2 atoms confers the advantage that the Cz^{D} is well-separated so that any undesirable interaction is minimised.

BTM peptides which are not commercially available can be synthesised by solid phase peptide synthesis as described in P. Lloyd-Williams, F. Albericio and E. Girald; Chemical Approaches to the Synthesis of Peptides and Proteins, CRC Press, 1997.

The imaging agents can be prepared as follows:

In order to facilitate conjugation of the Cz^{D} to the BTM, the Cz^{D} suitably has attached thereto a reactive functional group (Q^{a}). The Q^{a} group is designed to react with a complementary functional group of the BTM, thus forming a covalent linkage between the Cz^{D} and the BTM. The complementary functional group of the BTM may be an intrinsic part of the BTM, or may be introduced by use of derivatisation with a bifunctional group as is known in the art. Table 1 shows examples of reactive groups and their complementary counterparts:

TABLE 1
Reactive Substituents and Complementary Groups Reactive Therewith.

| Reactive Group (Q^{a}) | Complementary Groups |
|---|---|
| activated ester | primary amino, secondary amino |
| acid anhydride, acid halide. | primary amino, secondary amino, hydroxyl |
| isothiocyanate | amino groups |
| vinylsulfone | amino groups |
| dichlorotriazine | amino groups |
| haloacetamide, maleimide | thiol, imidazole, hydroxyl, amines, |
| | thiophosphate |
| carbodiimide | carboxylic acids |
| hydrazine, hydrazide | carbonyl including aldehyde and ketone |
| phosphoramidite | hydroxyl group |
| azide | alkyne |
| alkyne | azide |

By the term “activated ester” or “active ester” is meant an ester derivative of the carboxylic acid which is designed to be a better leaving group, and hence permit more facile reaction with nucleophiles, such as amines. Examples of suitable active esters are: N-hydroxysuccinimide (NHS), pentafluorophenol, pentafluorothiophenol, para-nitrophenol and hydroxybenzotriazole. Preferred active esters are N-hydroxysuccinimide or pentafluorophenol esters.

When Q^{a} is an azide or alkyne, the conjugation reaction involves “click chemistry” forming a triazole ring. Details of click chemistry as used in conjugate formation are described in: “Synthesis and Functionalization of Biomolecules via Click Chemistry, C. Schilling et al, Chapter 15 pages 355-378 in “Click Chemistry for Biotechnology and Materials Science” [J. Lahann (Ed), Wiley (2009)]. Further approaches to functionalising biological targeting molecules with alkyne or azide groups are described by Nwe et al [Cancer Biother. Radiopharm., 24(3), 289-302 (2009)]. Li et al provide the synthesis of a compound of the type N₃-L¹—CO₂H, where L¹ is —(CH₂)4- and its use to conjugate to amine-containing BTMs [Bioconj. Chem., 18(6), 1987-1994 (2007)]. Hausner et al describe related methodology for N₃-L¹-CO₂H, where L¹ is —(CH₂)₂- [J.Med.Chem., 51(19), 5901-5904 (2008)]. De Graaf et al [Bioconj.Chem., 20(7), 1281-1295 (2009)] describe non-natural amino acids having azide side chains and their site-specific incorporation in peptides or proteins for subsequent click conjugation.

Examples of functional groups present in BTM such as proteins, peptides, nucleic acids carbohydrates and the like, include: hydroxy, amino, sulfhydryl, carbonyl (including aldehyde and ketone) and thiophosphate. Suitable Q^{a} groups may be selected from carboxyl; activated esters; isothiocyanate; maleimide; haloacetamide; hydrazide; vinylsulfone, dichlorotriazine and phosphoramidite. Preferably, Q^{a} is: an activated ester of a carboxylic acid, an isothiocyanate, a maleimide or a haloacetamide.

When the complementary group is an amine or hydroxyl, Q^{a} is preferably an activated ester, with preferred such esters as described above. A preferred such substituent on the Cz^{D} is the activated ester of a 5-carboxypentyl group. When the complementary group is a thiol, Q^{a} is preferably a maleimide or iodoacetamide group.

General methods for conjugation of optical dyes to biological molecules are described by Licha et al [Topics Curr.Chem., 222, 1-29 (2002); Adv.Drug Deliv.Rev., 57, 1087-1108 (2005)]. Peptide, protein and oligonucleotide substrates for use in the invention may be labelled at a terminal position, or alternatively at one or more internal positions. For reviews and examples of protein labelling using fluorescent dye labeling reagents, see “Non-Radioactive Labelling, a Practical Introduction”, Garman, A. J. Academic Press, 1997; “Bioconjugation—Protein Coupling Techniques for the Biomedical Sciences”, Aslam, M. and Dent, A., Macmillan Reference Ltd, (1998). Protocols are available to obtain site specific labelling in a synthesised peptide, for example, see Hermanson, G. T., “Bioconjugate Techniques”, Academic Press (1996).

Preferably, the method of preparation of the imaging agent comprises either:
(i) reaction of an amine functional group of a BTM with a compound of formula Y¹-(L)ₙ-[Cz^{D}]; or
(ii) reaction of a carboxylic acid or activated ester functional group of a BTM with a compound of formula Y²-(L)ₙ-[Cz^{D}];
(iii) reaction of a thiol group of a BTM with a compound of formula

Y³-(L)ₙ-[Cz^{D}];
wherein BTM, M^{IG}, L, n and Cz^{D} are as defined above, and
Y¹ is a carboxylic acid, activated ester, isothiocyanate or thiocyanate group;
Y² is an amine group;
Y³ is a maleimide group.

Y² is preferably a primary or secondary amine group, most preferably a primary amine group. In step (iii), the thiol group of the BTM is preferably from a cysteine residue.

In steps (i) to (iii), the BTM may optionally have other functional groups which could potentially react with the Cz^{D} derivative, protected with suitable protecting groups so that chemical reaction occurs selectively at the desired site only. By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Amine protecting groups are well known to those skilled in the art and are suitably chosen from: Boc (where Boc is tert-butyloxycarbonyl), Fmoc (where Fmoc is fluorenylmethoxycarbonyl), trifluoroacetyl, allyloxycarbonyl, Dde [i.e. 1(4,4-dimethyl-2,6-dioxocyclohexylidene)ethyl] or Npys (i.e. 3-nitro-2-pyridine sulfenyl). Suitable thiol protecting groups are Trt (Trityl), Acm (acetamidomethyl), t-Bu (tert-butyl); test:Butythio, methoxybenzyl, methoxybenzyl or methylbenzyl or Npys (3-nitro-2-pyridine sulfenyl). The use of further protecting groups are described in ‘Protective Groups in Organic Synthesis’, Theodora W. Greene and Peter G. M. Wuts, (John Wiley & Sons, 1991). Preferred amine protecting groups are Boc and Fmoc, most preferably Boc. Preferred amine protecting groups are Trt and Acm.

The Cz^{D} dyes of the invention can be prepared as described in the Examples.

Methods of conjugating optical reporter dyes, to amino acids and peptides are described by Licha (vide supra), as well as Flanagan et at [Bioconj.Chem., 8, 751-756 (1997)]; Lin et al, [ibid, 13, 605-610 (2002)] and Zaheer [Mol.Imaging, 1(4), 354-364 (2002)]. Methods of conjugating the linker group (L) to the BTM employ analogous chemistry to that of the dyes alone (see above), and are known in the art.

In a second aspect, the present invention provides a pharmaceutical composition which comprises the imaging agent of the first aspect, together with a biocompatible carrier, in a form suitable for mammalian administration.

The “biocompatible carrier” is a fluid, especially a liquid, in which the imaging agent can be suspended or dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). Preferably the biocompatible carrier is pyrogen-free water for injection or isotonic saline.

The imaging agents and biocompatible carrier are each supplied in suitable vials or vessels which comprise a sealed container which permits maintenance of sterile integrity and/or radioactive safety, plus optionally an inert headspace gas (eg. nitrogen or argon), whilst permitting addition and withdrawal of SOlutionS by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour.

Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pharmaceutical compositions of the present invention preferably have a dosage suitable for a single patient and are provided in a suitable syringe or container, as described above.

The pharmaceutical composition may optionally contain additional excipients such as an antimicrobial preservative, pH-adjusting agent, filler, stabiliser or osmolality adjusting agent. By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, ie. methyl, ethyl, propyl or butyl paraben or mixtures thereof; benzyl alcohol; phenol, cresol; cetrimide and thiomersal. Preferred antimicrobial preservative(s) are the parabens.

The term “pH-adjusting agent” means a compound or mixture of compounds useful to ensure that the pH of the composition is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethypaminomethane], and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The pharmaceutical compositions of the second aspect may be prepared under aseptic manufacture (i.e. clean room) conditions to give the desired sterile, non-pyrogenic product. It is preferred that the key components, especially the associated reagents plus those parts of the apparatus which come into contact with the imaging agent (eg. vials) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise some components in advance, so that the minimum number of manipulations needs to be carried out. As a precaution, however, it is preferred to include at least a sterile filtration step as the final step in the preparation of the pharmaceutical composition. The pharmaceutical composition of the second aspect is preferably prepared from a kit, as described for the third aspect below.

In a third aspect, the present invention provides a kit for the preparation of the pharmaceutical composition of the second aspect, which comprises the imaging agent of the first aspect in sterile, solid form such that upon reconstitution with a sterile supply of the biocompatible carrier, dissolution occurs to give the desired pharmaceutical composition.

In that instance, the imaging agent, plus other optional excipients as described above, may be provided as a lyophilised powder in a suitable vial or container. The agent is then designed to be reconstituted with the desired biocompatible carrier to give the pharmaceutical composition in a sterile, apyrogenic form which is ready for mammalian administration.

A preferred sterile, solid form of the imaging agent is a lyophilised solid. The sterile, solid form is preferably supplied in a pharmaceutical grade container, as described for the pharmaceutical composition (above). When the kit is lyophilised, the formulation may optionally comprise a cryoprotectant chosen from a saccharide, preferably mannitol, maltose or tricine.

In a fourth aspect, the present invention provides a conjugate of Formula I:

[BTM]-(L)ₙ-Cz^{D} (I)
where: L, n, BTM and Cz^{D} and preferred aspects thereof are as defined in the first aspect.

The conjugates of the fourth aspect are useful in the preparation of both imaging agents and pharmaceutical compositions of the invention, comprising Cz^{D} dyes of Formulae II and IIa. Preferred aspects of the BTM, L, n and Cz^{D} dye of Formulae II and IIa are as described above. The conjugates can be prepared as described in the first and fifth aspects of the present invention.

In a fifth aspect, the present invention provides a functionalised dihydrocarbazolium dye)(Cz^{D} useful in the preparation of the conjugate of the fourth aspect, wherein the Cz^{D} is of Formula II or Ha as defined in the first aspect, and said Cz^{D} further comprises a group Q^{a}, where Q^{a} is a reactive functional group suitable for conjugation to a BTM.

The reactive “functional group” (Q^{a}) and preferred embodiments thereof are as described in the first aspect (above).

In a sixth aspect, the present invention provides a method of in vivo optical imaging of the mammalian body which comprises use of either the imaging agent of the first aspect or the pharmaceutical composition of the second aspect to obtain images of sites of localisation of the BTM in vivo.

By the term “optical imaging” is meant any method that forms an image for detection, staging or diagnosis of disease, follow up of disease development or for follow up of disease treatment based on interaction with light in the green to near-infrared region (wavelength 500-1200 nm). Optical imaging further includes all methods from direct visualization without use of any device and involving use of devices such as various scopes, catheters and optical imaging equipment, eg. computer-assisted hardware for tomographic presentations. The modalities and measurement techniques include, but are not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarization, luminescence, fluorescence lifetime, quantum yield, and quenching. Further details of these techniques are provided by: (Tuan Vo-Dinh (editor): “Biomedical Photonics Handbook” (2003), CRC Press LCC; Mycek & Pogue (editors): “Handbook of Biomedical Fluorescence” (2003), Marcel Dekker, Inc.; Splinter & Hopper: “An Introduction to Biomedical Optics” (2007), CRC Press LCC.

The green to near-infrared region light is suitably of wavelength 500-1200 nm, preferably of wavelength 600-1000 nm. The optical imaging method is preferably fluorescence endoscopy. The mammalian body of the sixth aspect is preferably the human body. Preferred embodiments of the imaging agent are as described for the first aspect (above). In particular, it is preferred that the Cz^{D} dye employed is fluorescent.

In the method of the sixth aspect, the imaging agent or pharmaceutical composition has preferably been previously administered to said mammalian body. By “previously administered” is meant that the step involving the clinician, wherein the imaging agent is given to the patient eg. as an intravenous injection, has already been carried out prior to imaging. This embodiment includes the use of the imaging agent of the first embodiment for the manufacture of a diagnostic agent for the diagnostic imaging in vivo of disease states of the mammalian body where the BTM is implicated.

A preferred optical imaging method of the sixth aspect is Fluorescence Reflectance Imaging (FRI). In FRI, the imaging agent of the present invention is administered to a subject to be diagnosed, and subsequently a tissue surface of the subject is illuminated with an excitation light - usually continuous wave (CW) excitation. The light excites the Cz^{D} dye of the imaging agent. Fluorescence from the imaging agent, which is generated by the excitation light, is detected using a fluorescence detector. The returning light is preferably filtered to separate out the fluorescence component (solely or partially). An image is formed from the fluorescent light. Usually minimal processing is performed (no processor to compute optical parameters such as lifetime, quantum yield etc.) and the image maps the fluorescence intensity. The imaging agent is designed to concentrate in the disease area, producing higher fluorescence intensity. Thus the disease area produces positive contrast in a fluorescence intensity image. The image is preferably obtained using a CCD camera or chip, such that real-time imaging is possible.

The wavelength for excitation varies depending on the particular Cz^{D} dye used, but is typically in the range 500-1200 nm for dyes of the present invention. The apparatus for generating the excitation light may be a conventional excitation light source such as: a laser (e.g., ion laser, dye laser or semiconductor laser); halogen light source or xenon light source. Various optical filters may optionally be used to obtain the optimal excitation wavelength.

A preferred FRI method comprises the steps as follows:
(i) a tissue surface of interest within the mammalian body is illuminated with an excitation light;
(ii) fluorescence from the imaging agent, which is generated by excitation of the Cz^{D}, is detected using a fluorescence detector;
(iii) the light detected by the fluorescence detector is optionally filtered to separate out the fluorescence component;
(iv) an image of said tissue surface of interest is formed from the fluorescent light of steps (ii) or (iii).

In step (i), the excitation light is preferably continuous wave (CW) in nature. In step (iii), the light detected is preferably filtered. An especially preferred FRI method is fluorescence endoscopy.

An alternative imaging method of the sixth aspect uses FDPM (frequency-domain photon migration). This has advantages over continuous-wave (CW) methods where to greater depth of detection of the dye within tissue is important [Sevick-Muraca et al, Curr.Opin.Chem.Biol., 6, 642-650 (2002)]. For such frequency/time domain imaging, it is advantageous if the Cz^{D} has fluorescent properties which can be modulated depending on the tissue depth of the lesion to be imaged, and the type of instrumentation employed.

The FDPM method is as follows:
(a) exposing light-scattering biological tissue of said mammalian body having a heterogeneous composition to light from a light source with a pre-determined time varying intensity to excite the imaging agent, the tissue multiply-scattering the excitation light;
(b) detecting a multiply-scattered light emission from the tissue in response to said exposing;
(c) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(d) generating an image of the tissue by mapping the heterogeneous composition of the tissue in accordance with the values of step (c).

The fluorescence characteristic of step (c) preferably corresponds to uptake of the imaging agent and preferably further comprises mapping a number of quantities corresponding to adsorption and scattering coefficients of the tissue before administration of the imaging agent. The fluorescence characteristic of step (c) preferably corresponds to at least one of fluorescence lifetime, fluorescence quantum efficiency, fluorescence yield and imaging agent uptake. The fluorescence characteristic is preferably independent of the intensity of the emission and independent of imaging agent concentration.

The quantifying of step (c) preferably comprises: (i) establishing an estimate of the values, (ii) determining a calculated emission as a function of the estimate, (iii) comparing the calculated emission to the emission of said detecting to determine an error, (iv) providing a modified estimate of the fluorescence characteristic as a function of the error. The quantifying preferably comprises determining the values from a mathematical relationship modelling multiple light-scattering behaviour of the tissue. The method of the first option preferably further comprises monitoring a metabolic property of the tissue in vivo by detecting variation of said fluorescence characteristic.

The optical imaging of the sixth aspect is preferably used to help facilitate the management of a disease state of the mammalian body. By the term “management” is meant use in the: detection, staging, diagnosis, monitoring of disease progression or the monitoring of treatment. The disease state is suitably one in which the BTM of the imaging agent is implicated. Imaging applications preferably include camera-based surface imaging, endoscopy and surgical guidance. Further details of suitable optical imaging methods have been reviewed by Sevick-Muraca et al [Curr.Opin.Chem.Biol., 6, 642-650 (2002)].

In a seventh aspect, the present invention provides a method of detection, staging, diagnosis, monitoring of disease progression or monitoring of treatment of a disease state of the mammalian body which comprises the in vivo optical imaging method of the sixth aspect.

The invention is illustrated by the non-limiting Examples detailed below. Example 1 provides the synthesis of a carbazolium dye precursor. Example 2 provides the synthesis of carbazolium dye precursor having an N-sulfoalkyl group (to improve water solubility). Example 3 provides the synthesis of carbazolium dye precursors having carboxyalkyl substituents (to facilitate conjugation of the dye to biological targeting moieties). Example 4 provides the synthesis of carbazolium dye precursors having both sulfoalkyl and carboxyalkyl substituents. Example 5 provides the synthesis of three dyes of the invention (Dye 1, Dye 2 and Dye 3) as prophetic examples based on an improved carbazolium dye synthesis. Example 6 provides evidence that dihydrocarbazolium dyes of the invention have suitable photophysical properties for in vivo optical imaging.

### Abbreviations.

BP: boiling point

CV: column volumes

DCM: Dichloromethane

DMF: Dimethylformamide

DMSO: Dimethylsulfoxide 6p HPLC: High performance liquid chromatography

LC-MS: Liquid chromatography mass spectroscopy

PBS: Phosphate-buffered saline.

RT: Room Temperature

TFA: Trifluoroacetic acid.

THF: Tetrahydofuran

TLC: Thin Layer Chromatography

### Example 1

### Synthesis of Carbazolium Precursor (Compound 1d).

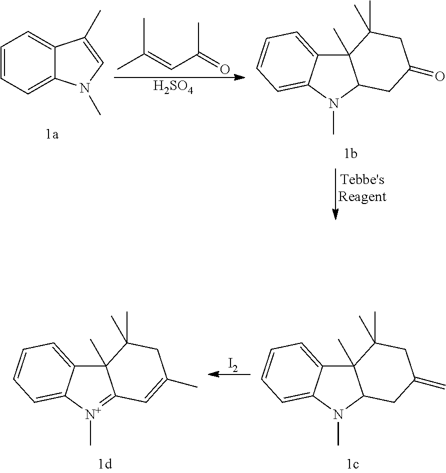

### (i) 1,3-Dimethylindole (Compound 1a).

1-Methyl-1-phenylhydrazine (6 g, 49.2 mmol) was slowly added to propionaldehyde (3.2 g, 9 mmol) in acetic acid (12 ml). Heat was evolved during the addition. The solution was heated in a CEM microwave reactor (200° C., 300 W, 1 minute hold time). The acetic acid was removed on a high vacuum rotary evaporator and the resulting black gum dissolved in DCM (20 ml), silica gel (50 g) added and the sample concentrated to dryness. Column chromatography (A=Petrol 40-60, B=DCM, 1-4 CV at 10% B, 13 CV 80% B, 40 g column). A fast running large peak was collected and concentrated give an impure yellow oil. The impure material was purified by fractional distillation, BP=190° C. at 100 Pa (1 mBar) where care had to be taken to separate the product from a higher boiling fraction to give the desired material (3,7 g, 44%).

¹H NMR (300 MHz, CDCl₃): δ (ppm) 2.33 (3H, s, methyl), 3.73 (3H, s, N-methyl), 6.82 (1H, s, 2-CH), 7.1 (1H, dd, indole), 7.21 (1H, dd, indole), 7.28 (1H, d, indole), 7.57 (1H, d, indole).
(ii) 4,4,4a,9-Tetramethyl-1 2,3,4,5 6-hexahydrocarbazol-2-one (Compound 1b).

1,3-Dimethylindole (Compound 1a; 200 mg, 1.38 mmol) was dissolved in acetonitrile (2 ml) and was cooled on a water ice bath. Sulfuric acid (97%, 0.2 ml) was added and one minute later mesityl oxide (405 mg, 4.14 mmol) added. Heat was evolved and stirring continued for half an hour after which the temperature was allowed to rise to RT over one hour. The reaction was added slowly with cooling and stirring to a suspension of NaHCO₃ (1.267 g) in water (10 ml) and was extracted with diethyl ether (3×20 ml) and dried over MgSO₄. Evaporation gave a pale yellow oil which was dissolved in diethyl ether (20 ml) and the solution dried onto silica gel (˜5 g). Column chromatography (A=Petrol 40-60, B=DCM, 1-4 CV 5% B to 10% B, 10 CV 100% B, 13 CV, 100% B, 120 g column) gave one main species which crystallized on standing to give the desired material (302 mg, 90%).

¹H NMR (CDCl₃): δ (ppm) 0.81 (3H, s, CH₃), 1.06 (3H, s, CH₃), 1.46 (3H, s, CH₃), 2.20 (1H, d, 3-CH), 2.32 (1H, d, 3-CH), 2.62 (3H, s, N—CH₃), 2.72 (2H, m, 1-CH₂), 3.55 (1H, dd, 9a-CH), 6.54 (1H, d, 8-CH), 6.76 (1H, dd, 6-CH), 7.06 (1H, d, 5-CH), 7.14 (1H, dd, 7-CH).
(iii) 2-Methylene-4,4,4a,9-tetramethyl-1,2,3,4,5,6,-tetrahydrocarbazole (Compound 1c).

Compound 1b (870 mg, 4.04 mmol) was dissolved in dry THF (2 ml) and was cooled to −40° C. Pyridine (10 μl) was added followed by a solution of Tebbe's reagent (0.5 M solution in toluene, 2 eq. 8.08 mmol, 16.16 ml) added over approximately one minute under nitrogen. The mixture was stirred for half an hour at −40° C. and was allowed to rise to RT over two hours. The reaction was quenched by adding sodium hydroxide solution (0.45 ml of 15% aqueous solution) to the cooled reaction mixture (heat and gas evolved). The dark green mixture was diluted with diethyl ether (60 ml) and filtered on a celite pad. Silica gel (˜20 g) was added and the solvent evaporated. Column chromatography (A=Petrol 40-60, B=DCM, 1-3 CV 0-5% B, 3-7 CV 5% B to 40% B, 7-9 CV 40% B to 100% B, 120 g column) gave the desired product as the major species (0.38 g, 39%).

NMR (CDCl₃): δ (ppm) 0.57 (3H, s, methyl), 0.96 (3H, s, methyl), 1.41 (3H, s, methyl), 1.84 (1H, d, CH 3), 2.28 (1H, d, CH 3), 2.43 (dd, 1H, H 1), 2.62 (1H, dd, H 1), 2.64 (3H, s, N-methyl), 2.99 (1H, dd, H 9a), 4.68 (1H, d, exo-CH₂), 4.81 (1H, d, exo-CH₂), 6.55 (1H, d, H 8), 6.74 (1H, dd, H 6), 7.05 (1H, d, H 5), 7.12 (1H, dd, H 7).
(iv) 2,4,4,4a, 9-Pentamethyl-4,4a-dihydro-3-H-carbazolium iodide (Compound 1d).

Compound 1c (300 mg, 1.24 mmol), iodine (2 eq. 631 mg, 2.49 mmol) and sodium iodide (615 mg, 4.1 mmol) in anhydrous methanol were heated at reflux under nitrogen for 2 hours. After cooling a precipitate formed which was collected by filtration, washed with ice cold water and dried under high vacuum, giving the desired product (110 mg, 23%). The filtrate was treated with a solution of sodium thiosulfate solution (0.2 ml saturated) and the product purified by semi-preparative HPLC (Phenomenex Luna C18(2) 150×21.2 mm, A=water, B=MeCN, 15 ml/min, λ=330 nm, 0-5 min 5% B, 12 min 70% B, 12-14 min 95% B, 14-18 min 5% B, tᵣ=11 min). Freeze-drying gave an off-white solid (122 mg, 27%). Total yield 50%.

¹H NMR (MeOH-d₃): δ (ppm) 0.59 (3H, s, methyl), 1.50 (3H, s, methyl), 1.54 (3H, s, methyl), 2.31 (3H, s, methyl), 2.45 (1H, d, H 3), 3.03 (1H, d, H 3), 3.99 (3H, s, N-methyl), 7.03 (1H, s, H 1), 7.65 (1H, dd), 7.65 (1H, dd), 7.75 (2H, m).

### Example 2

### Synthesis of N-Sulfoalkyl Carbazolium Precursor (Compound 2e).

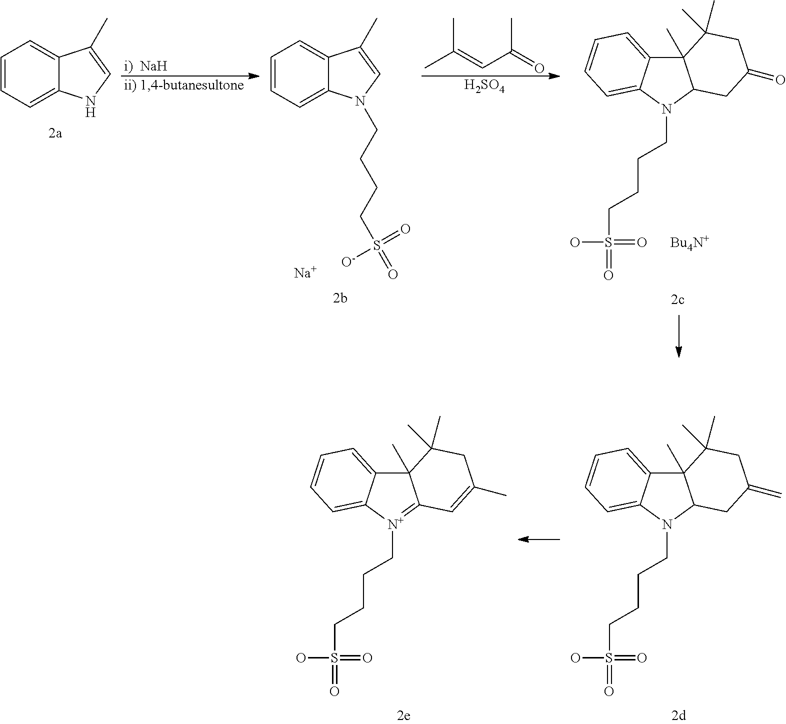

(i) 3-Methyl-1-(4-sulfobutyl)indole (Compound 2b).

3-Methylindole (Compound 2a; 311 mg, 2.38 mmol) was dissolved in dry DMF (5 ml) and sodium hydride (97 mg, 4.04 mmol, 1.7 eq.) (washed with dry diethyl ether) was added as a suspension in DMF (dry, 2 ml) in one portion. The fine suspension was stirred at RT for 30 minutes and 1,4-butanesultone (324 mg, 2.38 mmol) was added. The reaction was stirred for one hour. The reaction was then added to diethyl ether (100 ml) and the suspension stored at 4° C. for 16 hours. The supernatant was decanted off and the solid triturated with diethyl ether. The off-white solid was dried under high vacuum (410 mg, 61%). Proton NMR showed approximately 17 mol % DMF present. ¹H NMR (300 MHz, DMSO-d₆): δ (ppm) 1.54 (2H, m, N—CH₂CH₂CH₂CH₂—SO₃⁻), 1.74 (2H, m, N—CH₂CH₂CH₂CH₂—SO₃⁻), 2.24 (3H, s, methyl), 2.40 (2H, t, N—CH₂CH₂CH₂C₂—SO₃⁻), 4.10 (2H, t, N—CH₂CH₂CH₂CH₂—SO₃⁻), 6.98 (1H, dd, Ar), 7.11 (1H, dd, Ar), 7.12 (1H, s, 2-H), 7.39 (1H, d, ArH), 7.41 (1H, d, ArH).

(ii) 9-(4-Sulfobutyl)-4,4,4a-trimethyl-1,2,3,4,5,6-hexahydrocarbazol-2-one tetrabutylammonium salt (Compound 2c).

Compciurid 2b (2.0 g, 6.92 mmol) was suspended in acetonitrile (150 ml) and was warmed to 70° C. followed by sonication and further warming until most of the material had dissolved. Mesityl oxide (2 eq. 1.36 g, 13.84 mmol) was added followed by the slow addition of sulfuric acid (97%, 1 ml). The reaction was stirred at RT under nitrogen for 16 hours. Mesityl oxide (1 ml) was added followed by sulfuric acid (97%, 0.5 ml) and stirring was continued for 24 hours.

The reaction solution was concentrated under vacuum to give an oil, then water (80 ml) was added. Tetra-n-butylammonium hydroxide (solid) was added in small portions with stirring until the pH was approximately 7 and the solution was extracted with ethyl acetate (4×50 ml). The washings were not dried but were concentrated to give a yellow oil (3.5 g) which was dried under high vacuum for 16 hours. The oil was dissolved in DCM (80 ml) and dried onto silica gel (approximately 50 g). Column chromatography (A=DCM, B=10% methanol/DCM, C=methanol, 0-2 CV 100% A, 2-10 CV 0-100% B, 10-12 CV 100% B, 12-17CV 0-20% C, 17-20 CV 20% C, 330 g column) gave a range of fractions. Three main fractions eluting after 12 CV were shown to contain the desired product and some starting material as different salts. The slowest running fraction was shown by proton NMR to contain the desired product as the tetrabutylammonium salt as a clean material (0.87 g, 21%).

¹H NMR (300 MHz, CDCl₃): δ (ppm) 0.80 (3H, s, methyl), 1.00 (12H, t, NCH₂CH₂CH₂C₃), 1.06 (3H, s, methyl), 1.45 (8H, tt, NCH₂CH₂C₂CH₃), 1.46 (3H, s, methyl), 1.55 (2H, m, N-CH₂CH₂CH₂CH₂-SO₃), 1.6 (8H, m, NCH₂CH₂CH₂CH₃), 1.82 (2H, tt, N—CH₂CH₂CH₂CH₂-SO₃⁻), 2.20 (2H, 2×d overlapping, H 3 a/b system), 2.67 (2H, m, H 1 a/b system), 2.80 (2H, 2×d, N—CH₂CH₂CH₂—SO₃⁻), 3.0 (2H, m, N—CH₂CH₂CH₂CH₂—SO₃⁻), 3.32 (8H, t, NCH₂CH₂CH₂CH₃), 3.61 (1H, dd, 9a-H), 6.44 (1H, d, 8-H), 6.65 (1H, dd, 6-H), 7.00 (1H, d, 5-H), 7.06 (1H, dd, 7-H).
(iii) 2-Methylene-9-(4-sulfobutyl)-4,4,4a-trimethyl-1,2,3,4,5,6-hexahydrocarbazole tetrabutylammonium salt (Compound 2d).

Compound 2c (200 mg, 0.33 mmol) was dried using a toluene azeotrope evaporation (dry toluene, 3×20 ml) and was dissolved in THF (dry, 20 ml). The solution was cooled to −40° C. using a cardice/acetone bath. Tebbe's reagent (0.5 M solution in toluene, 1 mmol, 2 ml, 3 eq.) was added slowly over ca. 2 minutes. The reaction was stirred for 30 minutes at −40 ° C. under nitrogen and was allowed to rise to RT stirring for a further 30 minutes (1.5 h total reaction time). A solution of tetrabutylammonium hydroxide (3 eq. 1 mmol) in water (3 ml) was added slowly with cooling (vigorous reaction) and the mixture stored at −15 ° C. overnight. To the RT mixture DCM (50 ml) was added and the mixture filtered on a glass frit. Water was removed from the filtrate using a phase separator cartridge and the DCM solution concentrated to dryness to give an orange oil. The crude product was dissolved in DCM (15 ml) and a solution of tetrabutylammonium hydroxide (1 ml of 1 M solution in methanol) was added. The solution was dried onto silica gel (˜5 g). Column chromatography (A=DCM, B=10% methanol/DCM, C=methanol, 0-1 CV 100% A, 1-8 CV 0-100% B, 8-10 CV 100% B, 10-17CV 100% B to 20% C, 16-17% C 20% C, 40 g column) gave various peaks the slowest running proving to be the desired product (140 mg, 70%) but also contained 20-30% of the starting material ketone as an impurity. The impure material was used in the next step.

¹H NMR (CDCl₃): δ (ppm) 0.55 (3H, s, methyl), 0.90 (3H, s, methyl), 1.00 (12H, t, NCH₂CH₂CH₂CH₃), 1.34 (3H, s, methyl), 1.45 (8H, tt, NCH₂CH₂CH₂CH₃), 1.55 (2H, m, N—CH₂CH₂CH₂CH₂—SO₃⁻), 1.6 (8H, m, NCH₂CH₂CH₂CH₃), 1.90 (2H, m, N—CH₂CH₂CH₂C₂—SO₃⁻), 2.20 (2H, m, N—CH₂CH₂CH₂CH₂—SO₃⁻), 2.35 (1H, d, H 3), 2.55 (1H, d, H 3), 2.80 (3H, m, H 1 and H 9a), 3.0 (2H, m, N—CH₂CH₂CH₂CH₂—SO₃), 3.32 (8H, t, NCH₂CH₂CH₂CH₃), 4.63 (1H, s, exo-methylene), 4.76 (1H, s, exo-methylene), 6.42 (1H, d), 6.62 (1H, dd), 7.00 (1H, d), 7.05 (1H, dd).
(iv) 9-Sulfobutyl-2,4,4,4a,-Tetramethyl-4,4a-dihydro-3-H-carbazolium tetrabutyl ammonium salt (Compound 2e).

Compound 2d (100 mg, 0.17 mmol) was dissolved in methanol (dry, 10 ml) and iodine (1 eq. 41 mg, 0.17 mmol) was added. The solution was heated at reflux for 35 minutes. The reaction solution was concentrated to approximately half of the volume and water added slowly to give approximately 10 ml total volume. Purification was by semi-preparative HPLC (Phenomenex Luna C18(2) 150×21.2 mm, A=water, B=MeCN, 15 ml/min, λ=330 nm, 0-2 min 5% B, 12 min 70% B, 12-14 min 95% B, 14-18 min, 5% B, tᵣ=20.9 min). The collected fractions were concentrated and then freeze dried. Mass yield=19 mg.

¹H NMR (MeOH-d₄): δ (ppm) 0.59 (3H, s, methyl), 1.02 (3H, t, methyl), 1.4 (q), 1.5 (m), 1.65 (2H, m), 1.9 (2H, m), 2.05 (2H, m), 2.30 (2H, m), 2.45 (2H, 2×d), 2.9 (m), 3.05 (2H, 2×d), 3.25 (2H, m), 4.45 (m), 7.12 (1H, s), 7.64-7.8 (4H, m).

### Example 3

### Synthesis of Carboxypropyl-functionalised Carbazolium Precursors (Compounds 3e and 3f).

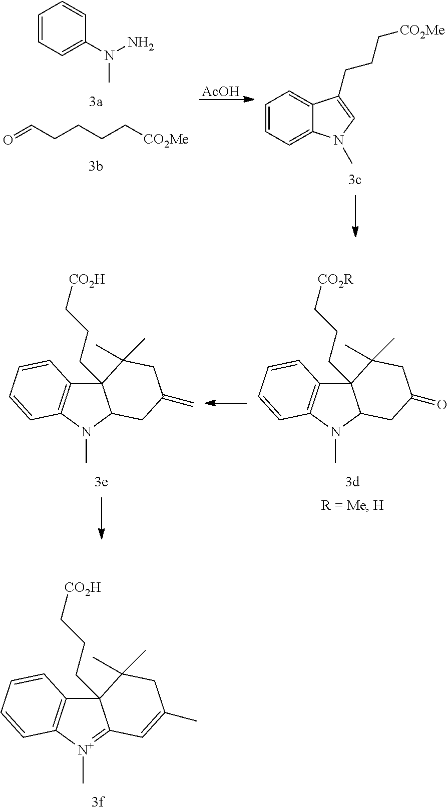

(i) 3-(3-Methox ycarbonylprop-l-v1)-1-m ethylindo le (Compound 3c).

1-Methyl-1-phenylhydrazine (3a; 845 g, 6.93 mmol) was dissolved in acetic acid (10 ml) and adipic semialdehyde methyl ester (3b; 997 mg, 6.93 mmol) added dropwise (heat evolved). The solution was heated in at reflux under nitrogen for 3 h. The acetic acid was removed on a high vacuum rotary evaporator and the resulting thick oil dissolved in DCM (20 ml), silica gel (˜10 g) added and the sample concentrated to dryness. Column chromatography (A=Petrol 40-60, B=DCM, 1-5 CV 10% to 30% B, 5-10 CV 30% to 100% B, 10-14 CV 100% B, 120 g column). A single large peak was collected and concentrated to give an oil (1.1 g, 69%).

¹H NMR (CDCl₃): δ (ppm) 2:06 (2H, tt, COCH₂CH₂CH₂), 2.39 (2H, t, COCH₂CH₂CH₂), 2.79 (2H, t, COCH₂CH₂CH₂), 3.66 (3H, s, OMe), 3.74 (3H, s, N-methyl), 6.84 (1H, s, H 2), 7.10 (1H, dd, Ar), 7.26 (1H, dd, Ar), 7.27 (1H, d, Ar), 7.60 (1H, d, Ar).
(ii) 4a-(3-Carboxyprop-1-yl)-4,4,9-trimethyl-1,2,3,4,5,6-tetrahydrocarbzol-2-one (Compound 3d).

Compound 3c (250 mg, 1.08 mmol) and mesityl oxide (106 mg, 1.08 mmol) were dissolved in acetonitrile (5 ml) and cooled to 0° C. The solution was degassed (vacuum/nitrogen gas cycled×3) and placed under nitrogen. Sulfuric acid (97%, 0.5 ml) was added dropwise and the reaction stirred at 0° C. The reaction was allowed to rise to RT and was stirred for 48 hours. The pH of the reaction was adjusted to approximately 5 using 10% aqueous K₂CO₃ and the solution extracted with diethyl ether (3×20 ml), the extracts were dried over MgSO₄ and concentrated to give a pink coloured oil. The product was purified by column chromatography (A=DCM, B=10% MeOH/DCM, 1-8 CV 0% to 50% B, 8-12 CV 50% to 100% B, 12-18 CV 100% B, 40 g column). One fraction was eluted which was shown to be a mixture of the desired product and the corresponding carboxylic acid. This mixture was separated by repeating the column chromatography procedure. Two fractions were obtained. The faster running species was the desired product (0.25 g, 70%) and the slower running species was the corresponding free acid (0.1 g, 29%).

¹H NMR (CDCl₃): δ (ppm) 0.87 (3H, s, methyl), 1.06 (3H, s, methyl), 1.30 (1H, m, COCH₂CH₂CH₂), 1.46 (1H, m, COCH₂CH₂CH₂), 1.66 (1H, m, COCH₂CH₂CH₂), 2.0 (1H, m, COCH₂CH₂CH₂), 2.20 (2H, 2×d overlapping, H 3), 2.33 (2H, t, COCH₂CH₂CH₂), 2.62 (3H, s, N-methyl), 2.71 (2H, 2×d, H 1), 3.58 (1H, dd, H 9a), 3.64 (3H, s, OMe), 6.50 (1H, d, H 8), 6.76 (1H, dd, H 6), 6.95 (1H, d, H 5), 7.12 (1H, dd, H7).
(iii) 4a-(3-Carboxyprop-1-yl)-2-methylidene-4,4,9-trimethyl-1,2,3,4,5,6-tetrahydrocarbzole (Compound 3e).

This compound is prepared using the same procedure as that for Compound 2d using Compound 3d.

(iv) 4a-(3-Carboxyprop-1-yl)-2-methylidene-2,4,4,9-tetramethyl- 4,4a-dihydro-3H-carbzolium iodide (Compound 3f).

This compound is prepared using the same procedure as that for Compound 2e using Compound 3e.

### Example 4

### Synthesis of Carboxyalkyl- and Sulfoalkyl-functionalised Carbazolium Precursors (Compounds 4f and 4g).

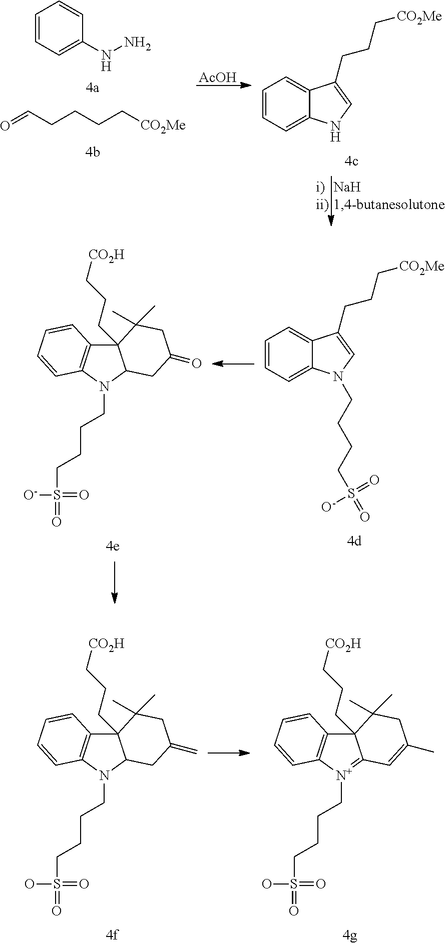

(i) 3-(3-Methoxycarbonylprop-1-yl)indole (Compound 4c).

Adipic semialdehyde methyl ester (4b; 5.92 g, 41.1 mmol) was added dropwise to a solution of phenylhydrazine (4a; 4.04 g, 37.4 mmol) in acetic acid and was heated under reflux for 1 h. The mixture was allowed to cool, then the solvent was removed in vacuo to afford a dark orange solid. The material was purified using flash chromatography (100% DCM eluent→5% MeOH). The crude compound was loaded onto the column as a liquid injection. The material was obtained as two fractions F3-20 (pure by ¹H NMR) 1.48 g, F21-31 (slightly impure˜95% by ¹H NMR) 0.978 g. Compound 4c was obtained in 31% yield.

¹H NMR (300 MHz; CDCl₃) δ (2H, quintet, J=7.7 Hz, CH₂CH₂CH₂), 2.35 (2H, t, J=7.3 Hz, CH₂), 2.75 (2H, t, J=7.02 Hz, CH₂CO₂Me), 3.61 (3H, s, OMe), 6.81 (1H, d, J=2.1 Hz, NHCH), 7.07 (1H, ddd, J=8.3 Hz, 7.3 Hz and 1.5 Hz, ArCH), 7.14 (1H, ddd, J=8.2 Hz, 7.05 Hz and 1.2 Hz, ArCH), 7.23 (1H, d, J=7.5 Hz, ArCH), 7.57 (1H, d, J=7.6 Hz, ArCH), 10.77 (1H, br s, NH).
(ii) 3-(3-Methoxycarbonvlprop-1-yl)-1-(4-sulfobutyl)indole (Compound 4d).

Compound 4c (4.0 g, 20.7 mmol) was dissolved in DMF (50 ml) and sodium hydride (0.81 g, 20.2 mmol) was added as a suspension in DMF (20 ml) in one portion. The dark purple suspension was stirred at RT for 6 hours. A 0.5 molar equivalent of NaH and the sultone was added and the reaction left to stir for a further 6 hours at room temperature. The reaction mixture was added to diethyl ether and the resulting suspension stirred at room temperature overnight. The mixture was filtered and the solid that had been collected rapidly turned into a gum. The gum was washed/dissolved in methanol and evaporated to dryness to form a brittle orange foam (3.88 g, 56%).

¹H NMR (DMSO; 300 MHz), 1.49-1.61 (2H, m, CH₂), 1.73-1.91 (2H, m, CH₂), 2.32 (2H, t, J_{HH}7 Hz, CH₂), 2.43 (2H, t, J=7 Hz, CH₂), 2.68 (2H, t, J=7 Hz, CH₂), 4.09 (2H, t, J=7 Hz, CH₂), 6.98 (1H, dt, J=7 Hz, J=0.6 Hz, ArCH), 7.10 (2H, dt, J=7 and 0.6 Hz, ArCH), 7.13 (1H, s, ArCH), 7.41 (1H, d, J=8 Hz, ArCH), 7.50 (1H, d, J=7 Hz, ArCH).
(iii) 4a-(3-Carboxyprop-1-yl)-4,4-dimethyl-9-(4-sulfobutyl)-1,2,3,4,5, 6-tetrahydrocarbazol-2-one (Compound 4e).

This compound is prepared analogously to Compound 2c using Compound 4d and mesitylene oxide.

(iv) 4a-(3-Carboxyprop-1-yl)-4,4-dimethyl-2-methylidene-9-(4-sulfobutyl)-1,2,3,4,5,6-tetrahydrocarbazole (Compound 41).

This compound is prepared analogously to Compound 2d using Compound 4e.

(v) 4a-(3-Carboxyprop-1-yl)-2,4,4-trimethyl-9-(4-sulfobutyl)-4,4a,-dihydro-3H-carbazolium iodide (Compound 4g).

This compound.is prepared⁻analogously to Compound 2e using Compound 4f.

### Example 5

### Synthesis of Carbazolium Dyes (Dye A, plus Dyes 1 to 3).

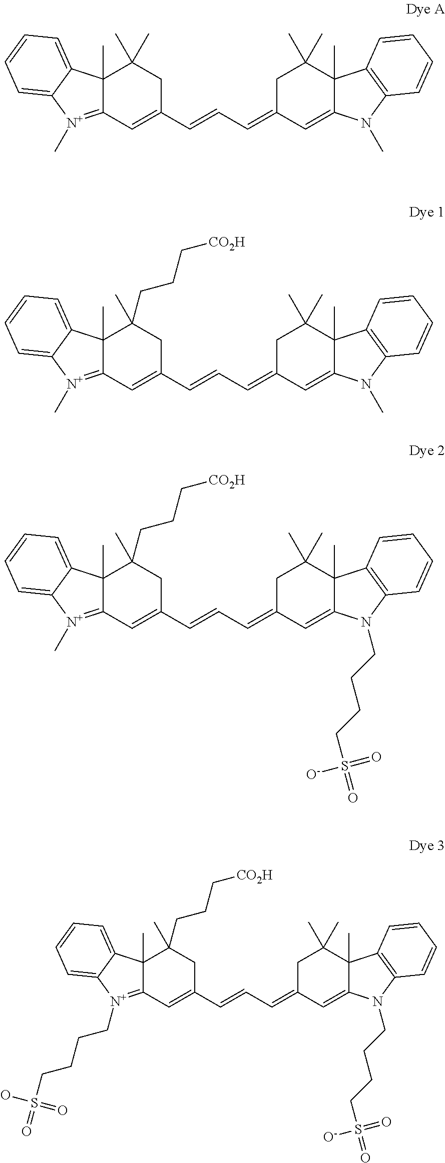

(i) 4,4,4a,9-Tetramethyl-2-(3-(1E-4,4,4a,9-tetramethyl-4,4a-dihydro-3H-carbazol-2-ylidenyl)prop-1-enyl)-4,4a-dihydro-3H-carbazolium iodide (Dye A; prior art).

This is an improved synthesis over that of U.S. Pat. No. 5,892,056:

Compound 1d from Example 1 (110 mg, 0.30 mmol) and triethylorthoformate (3 eq. 133 mg, 0.90 mmol) were heated at reflux in pyridine under nitrogen in the dark for 4h. The pyridine was removed under vacuum and the crude product dissolved in methanol (3 ml) whilst minimising exposure to light. Semi-preparative HPLC (Phenomenex Luna C18(2) 150×21.2 mm, A=0.5% TFA/water, B=0.5% TFA/MeCN, 15 ml/min, λ700 nm, 0-1 min 10% B, 13 min 95% B, 13-17 min 95% B, 17-20 min, 10% B, tᵣ=13.7 min). The product peaks over a number of runs were collected manually into scintillation vials which were immediately placed into a freezer (also in the dark). The fractions were combined and the solvents removed to give a deep blue/gold film. The product was dissolved in toluene (10 ml) and the solvent removed under vacuum. Finally the product was dried under high vacuum for 24 hours. Proton NMR and LCMS showed that the product was impure. The purification step was repeated as described above over two runs (3 mg, 2%).

¹H NMR (DMSO-d₆): δ (ppm) 0.67 (6H, s, methyl), 1.40 (6H, s, methyl), 1.56 (6H, s, methyl), 2.80 (2H, dd, H 3), 3.0 (2H, dd, H 3), 3.52 (6H, s, N-methyl), 6.29 (2H, s, H 1), 6.40 (2H, d, methine), 7.23 (2H, dd), 7.26 (2H, d), 7.40 (2H, dd), 7.55 (2H, d, H 8), 7.88 (1H, t, central methine H):
LCMS: m/z calculated for C₃₅H₄₁N₂ 489.3, found 489 [M]⁺

Dye 1 is prepared analogously to Dye A using one molar equivalent of each of Compound 1d and Compound 3f.

Dye 2 is prepared analogously to Dye A using one molar equivalent of each of Compound 2e and Compound 3f.

Dye 3 is prepared analogously to Dye A using one molar equivalent of each of Compound 2e and Compound 4g.

### Example 6

### Photophysical Properties of Carbazolium Dyes.

(a) Absorbance and fluorescence of Dye A vs Cyanine Dye Cy7.

0.1 to 0.2 mg vials of the dyes were dissolved initially in 100 μl DMSO and then diluted in PBS to the specified concentrations. Absorbance measurements were performed on 2.5 μM solutions, with a HP 8452A Diode array spectrophotometer. Fluorescence measurements were performed on 250 nM solutions, with a Varian Cary Eclipse Fluorescence Spectrophotometer at standardised instrument settings (600V PMT setting).

The measured absorbance (at 746 nm) equates to 260,000/M/cm.

The measured absorbance (at 748 nm) equates to 250,000/M/cm.

The fluorescence spectra of FIGS. 2 and 4 are directly comparable, as both samples were acquired at the same vial concentration, dilution media (PBS) and instrument settings.

(b) Photobleaching.

2 ml of the diluted solutions were exposed to a Xenon light source (Karl Storz Xenon 175 Model 20 1321 20) under identical illumination conditions. The sample cuvettes were kept in a water bath at room temperature during exposure, to avoid sample heating. Fluorescence spectra from the samples were recorded at 0,1,2,3,5 and 10 minutes exposure. Spectra are given below:

What is claimed is:

1. An imaging agent suitable for in vivo optical imaging of the mammalian body which comprises a conjugate of Formula I:
[BTM]-(L)ₙ-Cz^{D} (I)
where:
BTM is a biological targeting moiety;
Cz^{D} is a dihydrocarbazolium dye of Formula II: 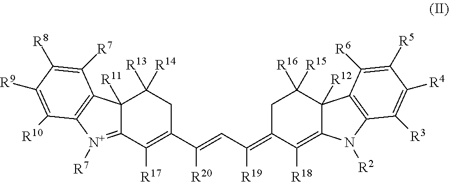
where:
R¹, R², and R¹¹ to R¹⁶ are each independently R^{a} groups,
R³ to R¹° are each independently H, —SO₃M¹,
—CO₂M¹, C₂₋₇ carboxyalkyl, C₁₋₄ hydroxyalkyl, or C₂₋₇ carboxamidoalkyl optionally substituted with 1 to 3 hydroxy groups, where M¹ is independently H or B^{c}, and B^{c} is a biocompatible cation;
R¹⁷ to R²⁰ are each independently H or an R^{a} group;
where R^{a} is C₁₋₄ alkyl, C₁₋₄ sulfoalkyl, C₂₋₇ carboxyalkyl or C₁₋₄ hydroxyalkyl;
L is a synthetic linker group of formula -(A)ₘ- wherein each A is independently —CR₂—, —CR═CR—, —CC—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
n is an integer of value 0 or 1;
with the proviso that the dihydrocarbazolium dye comprises at least 2 sulfonic acid substituents.

2. The imaging agent of claim 1, where R¹⁹═R²⁰═H.

3. The imaging agent of claim 1, where Cz^{D} has a total of 3 or 4 sulfonic acid substituents.

4. The imaging agent of claim 3, where the sulfonic acid substituents comprise 1 to 3 sulfoalkyl groups.

5. (canceled)

6. The imaging agent of claim 1, where one of R¹¹ and R¹² is C₁₋₄ sulfoalkyl or C₂₋₇ carboxyalkyl, and the other is CH₃.

7. The imaging agent of claim 1, wherein said Cz^{D} is a dihydrocarbazolium dye of Formula IIa: 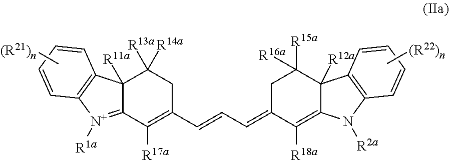
where:
R^{1a} and R^{2a} are each independently R^{b} groups;
R₁₁ₐ to R^{12a} are each independently CH₃ or an R^{b} group;
R^{13a} to R^{16a} are each independently CH₃, CH₂OH or C₂₋₅ carboxyalkyl;
R^{17a} and R^{18a} are each independently H or an R^{b} group;
R²¹ and R²² are each independently —SO₃M¹ or —CO₂M¹;
where R^{b} is C₁₋₄ sulfoalkyl or C₂₋₇ carboxyalkyl;
each n is independently 0, 1 or 2.

8. The imaging agent of claim 7, where at least one of R^{17a} and R^{18a} is H.

9. (canceled)

10. The imaging agent of claim 7, where one of R^{11a} and R^{12a} is an R^{b} group, and the other is CH₃.

11. The imaging agent of claim 1, where BTM is chosen from:
(i) a single amino acid;
(ii) a 3-100 mer peptide;
(iii) an enzyme substrate, an enzyme antagonist, an enzyme agonist, an enzyme inhibitor;
(iv) a receptor-binding compound;
(v) an oligonucleotide;
(vi) an oligo-DNA or oligo-RNA fragment.

12. (canceled)

13. The imaging agent of claim 11, where BTM is a 3-100 mer peptide and said imaging agent is of Formulae IVa or IVb:
[Cz^{D}]-(L)ₙ-[BTM]-Z² (IVa);
Z¹-[BTM]-(L)ₙ-[Cz^{D}] (IVb);
where:
Z¹ is attached to the N-terminus of the BTM peptide, and is H or M^{IG};
Z² is attached to the C-terminus of the BTM peptide and is OH, OB^{c}, or M^{IG}, where B^{c} is a biocompatible cation, and
M^{IG} is a metabolism inhibiting group which is a biocompatible group which inhibits or suppresses enzyme metabolism of the BTM peptide.

14. (canceled)

15. A pharmaceutical composition which comprises the imaging agent of claim 1 together with a biocompatible carrier.

16. (canceled)

17. A kit, which comprises the imaging agent of claim 1 in sterile, solid form.

18. (canceled)

19. A conjugate of Formula I:
[BTM]-(L)ₙ-Cz^{D} (I)
where:
BTM is a biological targeting moiety;
Cz^{D} is a dihydrocarbazolium dye of Formula II: 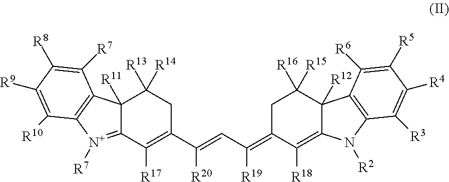
where:
R¹, R², and R¹¹ to R¹⁶ are each independently R^{a} groups,
R³ to R¹⁰ are each independently H, —SO₃M¹,
—CO₂M¹, C₂₋₇ carboxyalkyl, C₁₋₄ hydroxyalkyl, roxyalkyl, or C₂₋₇ carboxamidoalkyl optionally substituted with 1 to 3 hydroxy groups, where M¹ is independently H or B^{c}, and B^{c} is a biocompatible cation;
R¹⁷ to R²⁰ are each independently H or an R^{a} group;
where R^{a} is C₁₋₄ alkyl, C₁₋₄ sulfoalkyl, C₂₋₇ carboxyalkyl or C₁₋₄ hydroxyalkyl;
L is a synthetic linker group of formula -(A)ₘ- wherein each A is independently —CR₂—, —CR═CR—, —C═C—, —CR₂CO₂—, —CO₂CR₂—, —NRCO—, —CONR—, —NR(C═O)NR—, —NR(C═S)NR—, —SO₂NR—, —NRSO₂, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
n is an integer of value 0 or 1;
with the proviso that the dihydrocarbazolium dye comprises at least 2 sulfonic acid substituents.

20. A functionalised dihydrocarbazolium dye which comprises (i) a dihydrocarbazolium dye Cz^{D} of Formula II: 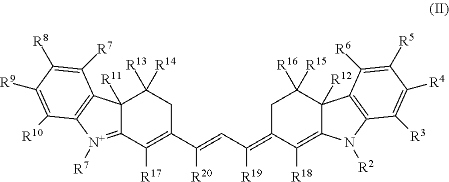
where:
R¹, R², and R¹¹ to R¹⁶ are each independently R^{a} groups,
R³ to R¹⁰ are each independently H, —SO₃M¹,
—CO₂M¹, C₂₋₇ carboxyalkyl, C₁₋₄ hydroxyalkyl, or C₂₋₇ carboxamidoalkyl optionally substituted with 1 to 3 hydroxy groups, where M¹ is independently H or B^{c}, and B^{c} is a biocompatible cation;
R¹⁷ to R²⁰ are each independently H or an R^{a} group;
where R^{a} is C₁₋₄ alkyl, C₁₋₄ sulfoalkyl, C₂₋₇ carboxyalkyl or C₁₋₄ hydroxyalkyl;
L is a synthetic linker group of formula -(A)ₘ- wherein each A is independently —CR₂—, —CR═CR—, —C═C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
n is an integer of value 0 or 1;
with the proviso that the dihydrocarbazolium dye comprises at least 2 sulfonic acid substituents;
or Formula IIa: 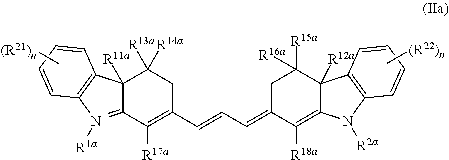
where:
R^{1a} and R^{2a} are each independently R^{b} groups;
R^{11a} to R^{12a} are each independently CH₃ or an R^{b} group;
R^{13a} to R^{16a} are each independently CH₃, CH₂OH or C₂₋₅ carboxyalkyl;
R^{17a} and R^{18a} are each independently H or an R^{b} group;
R²¹ and R²² are each independently —SO₃M¹ or —CO₂M¹;
where R^{b} is C₁₋₄ sulfoalkyl or C₂₋₇ carboxyalkyl;
each n is independently 0, 1 or 2
and (ii) a group Q^{a}, where Q^{a} is a reactive functional group suitable for conjugation to a BTM.

21. (canceled)

22. A method of in vivo optical imaging of the mammalian body which comprises use of either the imaging agent of claim 1 or the pharmaceutical composition of claim 15 to obtain images of sites of localisation of the BTM in vivo.

23-28. (canceled)

29. A method of detection, staging, diagnosis, monitoring of disease progression or monitoring of treatment of a disease state of the mammalian body which comprises the in vivo optical imaging method of claim 22.
